(19) United States
(12) Patent Application Publication
(10) Pub. No.: US 2005/0288250 A1
(43) Pub. Date: Dec. 29, 2005

(54) Novel use of carbohydrates and compositions

(75) Inventors: Nina Rautonen, Helsinki (FI); Juha Apajalahti, Helsinki (FI); Anu Kettunen, Espoo (FI); Osmo Siikanen, Virkkala (FI)

Correspondence Address: SCULLY SCOTT MURPHY & PRESSER, PC, 400 GARDEN CITY PLAZA, SUITE 300, GARDEN CITY, NY, 11530, US

(73) Assignee: Danisco A/S, Copenhagen K (DK)

(21) Appl. No.: 10/663,562

(22) Filed: Sep. 16, 2003

(30) Foreign Application Priority Data
Sep. 17, 2002 (FI) FI 200321660

(51) Int. Cl. A61K 31/715

(52) U.S. Cl. 514/54

(57) ABSTRACT

A process for preventing colon fermentation imbalance by the administration of a composition of a slowly fermented carbohydrate complex oligomer or polymer. A method of preparing the composition by nutritionally, nutraceutically and/or pharmacologically formulating the carbohydrate oligomer or polymer is an aspect of this process.

### FIELD OF INVENTION

This invention relates to a novel use of slowly fermented carbohydrates in the preparing of a composition for treating and preventing various diseases or disorders caused by imbalanced colon fermentation. The present invention also relates to compositions comprising a slowly fermented complex oligomer and polymer carbohydrate. The present invention is used in various nutritional, nutraceutical and pharmacological applications.

### BACKGROUND OF THE INVENION

The health and well being of people and animals can be positively or negatively influenced by the functioning of the colon fermentation and micro-organisms which pass and inhabit the gastrointestinal tract.

The intestinal lumen of animals comprises a large interface with the environment. The primary function of the small intestine is to absorb nutrients from the food. In order to allow maximal nutrient absorption, the contact area needs to be large. The mucosal membrane of the whole human intestine covers over 200 m² of area. Approximately 25 tons of food and approximately the same amount of drinks pass through the intestine during a lifetime. However, in addition to dietary compounds, the mucosal surface is exposed to various bacteria, viruses, parasites and fungi. Due to the monolayered epithelium covering the lumen, it is also an attractive gate for pathogens to the body. The state of the intestinal tract is a significant factor in many illnesses (e.g. infections, allergies and cancer).

The mammalian large intestine contains a substantial and diverse population of bacteria that is important to mammalian health. The beneficial microflora in the gut is able to salvage energy for the host through bacterial fermentation of undigested carbohydrates and proteins to provide short-chain fatty acids, which are then absorbed. The presumed beneficial genera, Bifidobacterium and Lactobacillus, both of which are saccharolytic, are thought to create conditions unfavorable for growth of potentially pathogenic species. Hence, bifidobacteria and lactobacillus are ingredients in various probiotic products, which are used to elevate the number of said microbes and lactic acid producing fermentation in the gut. On the other hand, various kinds of food and drink compositions, which include non-absorbable carbohydrates (oligosaccharides and polysaccharides), have been developed. These are called prebiotics and they are thought to stabilize the intestinal microbial balance in favour to the beneficial microbes. Furthermore, dietary fiber products have been used to treat constipation and to promote Taxation.

Acidosis is a phenomenon where colonic fermentation is disturbed. It is characterized by changes in the microbial community structure, especially by significant increase in numbers of lactobacilli, as well as lactic acid accumulation in the colon. As a result of an accumulation of lactic acid in the colon erosion of colonic mucosa can be detected, thus lactic acid can increase the risk for ulcerative colitis. Accumulation of lactic acid in the colon can be detected from the blood and can lead also to a condition called metabolic acidosis.

Acidosis results e.g. from an overload of rapidly fermented carbohydrates in the colon. Overflow of carbohydrates to the colon can be detected especially in patients with short gut syndrome or those with a resected small intestine, i.e. in persons with severely decreased capacity of absorption of nutrients. Conditions that result in an inflammation of the small intestine thereby shortening the villa and decreasing absorptive capacity, increase the risk of developing acidosis. Food allergy like celiac disease, or fulminant diarrhoea can increase the risk for developing acidosis. Furthermore acidosis can develop in sensitive persons after ingestion of large amounts of lactic acid bacteria. Yeast may also contribute to the development of acidosis. Lactic acid is also produced in large amounts by bacteria in the colon in persons with lactose intolerance, and can thus predispose to development of acidosis.

Inulin (Raftiline^{R}, provided by Orafti, Tienen, Belgium) has been shown to result in lactic acid accumulation in rodents (Apajalahti, J. H. A., et al., Appl. Environ. Microbiol., Vol 44, in press). Because inulin is a rapidly fermented prebiotic it can lead to situations where the microbial community balance is disturbed and accumulation of lactic acid takes place.

Complex carbohydrates are fermented differently depending on their chemical structure. Some complex carbohydrates, such as cellulose, are not fermented in the human intestine at all, whereas some complex carbohydrates, e.g. starch, are rapidly fermented. Slowly fermented complex oligomer and polymer carbohydrates are carbohydrates that have a very complex structure and which are not easily fermented.

Polydextrose is a slowly fermented complex polymer. It is a sugar polymer synthesized by random polymerisation of glucose, sorbitol and a suitable acid catalyst at a high temperature and partial vacuum. The term “polydextrose” is defined in greater detail later in this text. Polydextrose is widely used in various kinds of food products as a bulking agent and as a low-energy ingredient, replacing sugar and partially fat. Polydextrose is not digested or absorbed in the small intestine and a substantial portion is excreted in the feces. Polydextrose has been incorporated into a wide range of foods including baked goods, beverages, confectionary and frozen dairy desserts.

The beneficial effects of polydextrose on the intestinal tract have been described in Jie, Z et al., Am. J. Clin. Nutr. 72, pp. 1503-1509, 2000. The study shows that polydextrose had no significant effect of blood biochemistry indexes. Bowel function improved significantly and there were no abdominal distention, abdominal cramps, diarrhoea or hypoglycemia. Short-chain fatty acid production, notably that of butyrate, isobutyrate and acetate, increased with polydextrose digestion. Polydextrose intake caused substantial changes in fecal anaerobes. Bacteroides species decreased, whereas Lactobacillus and Bifidobacterium species increased. The study did not relate to the mechanism of digestion of polydextrose.

A comparative study with a low cholesterol diet, a high cholesterol diet and a high cholesterol diet supplemented by polydextrose showed that the diet with polydextrose significantly lowered fecal pH, and reduced the production of some carcinogens, like indole and p-cresol. (Endo, K. et al. Bifidobacteria Microflora, Vol. 10 (1), 53-64, 1991). U.S. Pat. No. 5,437,880 describes a health drink containing polydextrose; JP 2072842 describes a drink and food containing polydextrose as a dietary fiber, and EP 821885 describes a dairy powder containing polydextrose for promoting the function of the intestine.

None of the above mentioned documents indicate any effect of polydextrose or other slowly fermented complex oligomer or polymer carbohydrates on the prevention and treatment of acidosis or other disorders or diseases caused by imbalanced colon fermentation in the colon of a mammal.

Most present finctional food products (like prebiotics and probiotics) are targeted to stimulate lactic acid fermentation in the gastrointestinal tract and have been until now focusing on modulation of the intestinal motility function and the absorbance capacity, as well as on the modulation of the microflora balance in the intestinal tract, which plays an important role in combating against pathogens.

However, there is need for a convenient and effective way to prevent and treat disorders or diseases caused by imbalanced fermentation in the colon, such as acidosis, thereby promoting the health and well being of subjects such as mammals and other animals.

There is also a need for compounds capable of providing a sustained release of energy for use in functional foods. Some mammals have problems with the finctioning of the intestine due to e.g. short intestine, food allergy or damaged villa in the intestine. The absorption of nutrients in the small intestine is thereby deteriorated and the risk of inflammatory colon diseases and acidosis increases. Therefore, there is a need for an easy way to alleviate problems caused by poorly functioning intestines.

The beneficial effects of polydextrose on the intestinal tract are known, but the inventors of the present invention have now surprisingly found, that polydextrose as well as some other slowly fermented complex oligomer and polymer carbohydrates are effective in sustaining and controlling the fermentation throughout the colon of a subject. By sustaining and controlling the release of energy for bacterial fermentation such carbohydrates can act as a lactic acid accumulation preventing ingredient, thus preventing development of imbalanced colon fermentation. It has also been found that the slowly fermented complex oligomer and polymer carbohydrates can be used in a composition for increasing tolerance of probiotic lactic acid bacteria in sensitive people as well as for helping management of lactose intolerance, food allergy, celiac disease or inflammatory diseases in the colon.

Furthermore, the inventors have found that the slowly fermented complex oligomer and polymer carbohydrates, especially polydextrose, have beneficial effects in the prevention of an accumulation of lactic acid in the colon. They have also found that said carbohydrates and polyols have synergistically beneficial effects in preventing accumulation of lactic acid in the colon.

In addition to the prevention of accumulation of lactic acid in the colon, the inventors of the present application have found that the slowly fermented complex oligomer and polymer carbohydrates provide their beneficial effects not only in the proximal part of the colon, but that they provide these effects throughout the colon. Among these beneficial effects there are also a reduction of the putrefactive fermentation and a reduction of the pH throughout the colon. It has also been found that the slowly fermented complex oligomer and polymer carbohydrates are effective in increasing the amount of butyrate throughout the colon. Slowly fermented complex oligomer and polymer carbohydrates are additionally effective in balancing or normalizing the microbial community throughout the colon.

The preferred slowly fermented complex carbohydrate of the present invention is polydextrose. It is known that when polydextrose is consumed, a part of the carbohydrates of polydextrose origin exits from the colon. It is also known that fiber material in the colon as such improves the function of the colon the by its sheer bulk. However, the prior art knowledge of polydextrose digestion did not make it clear that polydextrose digestion extends throughout the colon. The inventors have found out that since polydextrose is feeding bacteria throughout the colon, it can be used in a different way than previously described prebiotics for treating and/or preventing diseases and/or disorders of the colon, especially the distal colon where harmful compounds tend to accumulate.

Hence, the present invention contributes to the overall health and well being of the intestinal tract by providing a method, which effectively prevents accumulation of lactic acid in the colon. The beneficial effect is observed throughout the colon fermentation.

### SUMMARY OF THE INVENTION

Accordingly, it is an object of the present invention to provide methods and compositions for strengthening and improving the health condition of the colon.

One aspect of the present invention is the use of carbohydrates as an active ingredient in the preparation of a composition for treating and/or preventing diseases and/or disorders caused by imbalanced colon fermentation. The composition of the present invention is prepared by formulating a slowly fermented complex oligomer and polymer carbohydrate into a nutritionally, nutraceutically and/or pharmacologically acceptable composition, said carbohydrate being effective in sustaining and controlling the fermentation throughout the colon. The preferred carbohydrates are effective in providing a sustained release of energy to the colon.

Another aspect of the present invention is the use of said carbohydrates in the preparation of a composition for preventing the accumulation or lactic acid throughout the colon.

A further aspect of the present invention is the use of said carbohydrates in the preparation of a composition for reducing the pH throughout the colon without accumulation of lactic acid.

Yet another aspect of the present invention is the use of said carbohydrates in the preparation of a composition for reducing the putrefactive fermentation throughout the colon. Putrefactive fermentation is based on degradation of proteins which leads to an abundance of toxic compounds and branched volatile fatty acids (VFAs). Branched VFAs can be used as biomarkers for such undesirable fermentation.

Yet another aspect of the present invention is the use of said carbohydrates in the preparation of a composition for increasing the amounts of butyrate throughout the colon.

Yet another aspect of the present invention is the use of said carbohydrates in the preparation of a composition for increasing the tolerance of probiotic lactic acid bacteria.

A further aspect of the present invention is the use of said carbohydrates in the preparation of a composition for facilitating the management of lactose intolerance.

A further aspect of the present invention relates to the use of said carbohydrates in the preparation of a composition for facilitating the management of food allergy.

Yet, a further aspect of the present invention relates to the use of said carbohydrates in the preparation of a composition for facilitating the management of the effects of celiac.

Yet, a further aspect of the present invention relates to the use of said carbohydrates in the preparation of a composition for reducing the risk of inflammatory diseases in the colon.

The present invention provides also a method for the therapeutic or prophylactic treatment of humans as well as animals suffering from or being subject to a risk of imbalanced colon fermentation. The method comprises administering a slowly fermented complex oligomer or polymer carbohydrate to the subject in an amount which is effective in sustaining and controlling the fermentation throughout the colon of said subject.

A further aspect of the present invention is a method, wherein the carbohydrate is administered in an amount which is effective in preventing the accumulation of lactic acid throughout the colon.

A further aspect of the present invention is a method, wherein said carbohydrate is administered in an amount which is effective in reducing the pH throughout the colon without accumulation of lactic acid.

A further aspect of the present invention is a method, wherein said carbohydrate is administered in an amount which is effective in reducing the putrefactive fermentation throughout the colon without accumulation of lactic acid.

A further aspect of the present invention is a method, wherein said carbohydrate is administered in an amount which is additionally effective in reducing the putrefactive fermentation throughout the colon and/or wherein the carbohydrate is administered in an amount which is additionally effective in increasing the amount of butyrate throughout the colon.

Yet, a further aspect of the present invention is a method, wherein said carbohydrate is administered in an amount which is effective in increasing the tolerance of probiotic lactic acid bacteria.

Yet, a further aspect of the present invention is a method, wherein said carbohydrate is administered in an amount which is effective in facilitating the management of lactose intolerance, food allergy and/or celiac disease.

Yet, a further aspect of the present invention is a method, wherein said carbohydrate is administered in an amount which is effective in reducing the risk of inflammatory diseases in the colon.

The methods of the present invention may take several embodiments. Additional objects, advantages and features of the various aspects of the present invention will become apparent from the following description of its preferred embodiments.

### BRIEF DESCRIPTION OF THE DRAWINGS

FIG. 1 depicts graphically the production of volatile fatty acids in a 4 stage colon fermentation simulator.

FIG. 2 depicts graphically the concentration of branched VFAs in the colon.

FIG. 3 depicts graphically the concentration of butyrate in relation to the proportion of branched VFAs in the colon.

### DETAILED DESCRIPTION OF THE INVENTION

The present inventors have surprisingly found that the administration of a slowly fermented complex oligomer or polymer carbohydrate very significantly prevents the colon fermentation from becoming imbalanced. The slowly fermented carbohydrate provides a sustained release of energy throughout the colon, which causes a shift in the microbial community in the gut and enhances the growth of microbes with a positive effect on the colon fermentation, thus causing all produced lactic acid to be further fermented.

The present invention relates to the use of a slowly fermented complex oligomer or polymer carbohydrate in the preparation of a composition for treating and/or preventing diseases and/or disorders caused by imbalanced colon fermentation. The slowly fermented complex oligomer or polymer carbohydrate of the present invention is formulated into a nutritionally, nutraceutically and/or pharmacologically acceptable composition. The carbohydrate is effective in sustaining and controlling the fermentation throughout the colon of a mammal which has several beneficial effects on the well being of the mammal. The present invention is especially effective since the positive effect of the carbohydrate can be obtained throughout the colon.

The villa in the mammalian colon may be shortened or damaged due to sensitivity to nutrients or due to an inflammation. In case the villa does not function properly, the absorption in the colon is disturbed, which may lead to an overload of fermentable energy in the colon. This may lead to imbalanced fermentation and cause disorders and diseases. Since the present invention provides a sustained and controlled fermentation throughout the colon, it can be used in the treatment and prevention of disorders and diseases caused by imbalanced colon fermentation.

The term “imbalanced” fermentation means that the fermentation is disturbed in the colon. The energy available for bacteria in the colon is not distributed evenly along the colon and, as a consequence, there are local exceptionally high or low amounts of metabolites from fermentation, such as lactic acid. The growth of the carbohydrate-fermenting microbes may vary from excessive to non-existent due to uneven availability of energy.

Imbalanced fermentation may cause various diseases or disorders, such as acidosis, inflammation, allergy, celiac disease, osteoporosis, etc due to the uncontrolled accumulation of lactic acid. It may also cause diarrhoea. However, diarrhoea can also be caused by very many other indications such as viruses or food poisoning. The present invention is meant to relate to only those diseases and disorders which are caused by imbalanced fermentation.

The term “sustained” in the present invention means that energy is supplied to the colon cells continuously nourishing the cells evenly in a prolonged period of time throughout the colon. The positive effect of the carbohydrates of the present invention is obtained since the carbohydrates are fermented slowly and continuously in the colon. Thus, the micro-organisms and the colon cells obtain energy steadily and function well throughout the colon, not only in part of the colon. When the carbohydrate energy supply to the micro-organisms is suitable, the fermentation functions properly without protein degradation and in a controlled and balanced manner. Thereby disorders or diseases caused by imbalanced fermentation are alleviated or prevented. It has now been found that in case the micro-organisms are not sustained properly, other energy sources (e.g. proteins) are used which may lead to imbalanced fermentation. Moreover, in case too much energy is released e.g. in the proximal part of the colon, a boost of fermentation is obtained which initially may have beneficial effects, but which gives rise to imbalance at a later stage in the colon.

The term “controlled” in the present invention means that energy is released evenly throughout the colon without major variations in the amount of available energy for the colon cells.

The term “colon cells” means the epithelial and immune cells in the colon of a mammal.

The term “nutraceutical” means a composition which is capable of providing not only a nutritional effect and/or a taste satisfaction, but is also capable of delivering a therapeutic (or other beneficial) effect to the consumer. The terms “pharmaceutical” and “nutritional” have the meanings generally applied to those terms.

The term “slowly fermented” in the present invention means that the carbohydrate is not rapidly utilized by the microorganisms in the gastrointestinal tract and that therefore a substantial amount of fermentable carbohydrate remains unfermented even at the distal end of the colon. Due to the slow fermentation the carbohydrates of the present invention provide a sustained release of energy to the microorganisms throughout the colon. In contrast, a rapidly fermented compound provides a boost of energy at the proximal end of the colon and is totally fermented before reaching the distal end of the colon.

The term “complex carbohydrate” in the present invention means a compound with a carbohydrate chemical structure which is complex from the point of view of fermentation. The complexity may be caused by the carbohydrate having different types of chemical bonds which require different types of enzymes for breaking down. The complex carbohydrate may also require fermentation by microorganisms which are not abundant in the colon. The complexity may be due to steric hindrances in the molecule or to physicochemical properties such as low solubility, crystallinity, particle size, etc. The complex carbohydrates useful in the present invention will be described in greater detail below.

The slowly fermented complex oligomer or polymer carbohydrates of the present invention provide a sustained release of energy in the colon and they are therefore also called “sustained release carbohydrates” in the present invention.

In the present invention a complex, sustained release carbohydrate is used for preventing the accumulation of lactic acid throughout the colon. The accumulation of lactic acid is one symptom of imbalanced colon fermentation. The accumulation of lactic acid in the colon indicates that the acid is not fermented by the micro-organisms in the colon. Bacteria metabolizing lactic acid further are inhibited by acidosis. Lactic acid is the strongest acid produced by intestinal bacteria (lowest pKa-value). Therefore, accumulation of lactic acid leads to an escalation of the accumulation and too rapid lowering of the pH in the proximal part of the colon. If the bacteria, which metabolize lactic acid further are killed as a result of the low pH, this may lead to acute acidosis.

In a preferred embodiment the sustained release carbohydrate is effective in reducing the pH throughout the colon of a mammal without accumulation of lactic acid. A decrease in the pH generally has beneficial effects on the colon. The present invention enables the reduction of pH without causing an accumulation of lactic acid, which on the other hand would have negative effects in the colon. Therefore the risk of infection by pathogenic attack is reduced and mineral absorption is improved, which again reduces osteoporosis.

In another preferred embodiment, an effective amount of a sustained release carbohydrate is used to prepare a composition, wherein the sustained release carbohydrate is additionally effective in reducing the putrefaction and its metabolites throughout the colon of a mammal. Thus, the formation of toxic compounds, which may cause cancer, is reduced in the colon.

Toxic compounds and biogenic amines which may be harmful in too large amounts result from putrefactive fermentation. Branched VFAs indicate the presence of putrefactive fermentation. Common branched VFAs are isobutyrate, isovalerate and 2-methylbutyrate. Readily digested prebiotics may promote the putrefactive fermentation if the substrate carbohydrate is consumed already in the proximal parts of the colon increasing numbers of bacteria rapidly. When the prebiotics have been consumed in the colon the bacteria in the distal colon start utilizing proteins as an energy source. The putrefactive fermentation is considered to have negative effects on the gut health, for example by increasing the risk for colon cancer. The reduced amount of branched VFAs in the colon provided by the use of sustained release carbohydrates according to the present invention indicates health-promoting, balanced bacterial metabolism in the colon.

In another preferred embodiment of the present invention, a sustained release carbohydrate is used to prepare a composition, in which the amount of the sustained release carbohydrate is additionally effective in increasing the amount of butyrate throughout the colon. Butyrate as such is considered beneficial for the intestine as it is an important energy source for colonocytes regulating cell growth and differentiation. Butyrate is also an interesting volatile fatty acid in terms of reducing colon cancer risk.

In another preferred embodiment a sustained release carbohydrate in a composition is effective in both reducing the putrefactive fermentation and increasing the amount of butyrate throughout the colon whereby dual benefits are obtained from one and the same composition.

Tolerance of probiotic lactic acid bacteria is increased by the use of a sustained release carbohydrate according to the present invention. A sustained release carbohydrate is also used for facilitating the management of lactose intolerance. Accordingly, lactic acid produced by the lactic acid bacteria does not harm since the accumulation of lactic acid is inhibited. The use of a sustained release carbohydrate according to the present invention also reduces problems encountered by food allergy, such as the effects of celiac disease in a mammal. Therefore, the present invention enables a balanced diet for mammals having disorders or diseases which otherwise limit their diet.

The present invention is also effective in reducing the risk of inflammatory diseases in the colon of a mammal.

In another preferred embodiment of the present invention, a sustained release carbohydrate is used to prepare a composition, in which the amount of the sustained release carbohydrate is additionally effective in balancing or normalizing the microbial community throughout the colon. Such a composition is beneficial especially after an antibiotic treatment or other disturbance in the intestinal tract since it expedites the recovery of a patient.

Slowly fermented complex oligomer or polymer carbohydrates of the present invention are carbohydrates that are not readily utilized by the micro-organisms of the gastrointestinal tract. Suitable complex oligomeric and polymeric carbohydrates have a complex chemical structure. Slowly fermented carbohydrates which are useful in the present invention may be selected e.g. by screening prospective carbohydrates in a batch fermentation with fecal bacteria. The carbohydrates which are fermented slowly by the bacteria are potentially useful in the present invention. To ascertain whether a potentially selected carbohydrate has sustained release properties the carbohydrate in question is then subjected to a colon simulation as described in greater detail below.

In a preferred embodiment of the invention the said carbohydrate is a sugar polymer. Sugar polymers of the present invention are sugar polymers which are resistant to enzyme digestion in the intestines and which are prepared by any of the processes described for polydextrose later in this text, using one or more sugars as the starting material. The term “sugar polymer” includes polydextrose, but also includes other food acceptable products in which other sugars are used in lieu of glucose in the polycondensation reaction. Thus, for example, it includes the products from the polymerization of sugars in the presence of sugar alcohol, as well as the purified products thereof. It also includes hydrogenated sugar polymers.

In another preferred embodiment of the present invention the carbohydrate comprises a slowly fermented carbohydrate, such as xanthan, alginate and/or xylooligomer or derivatives thereof.

Xanthan is an anionic bacterial polysaccharide composed of U-(1->4)-D-Glc(1->4)-beta-D-Glc (cellulosic) backbone with a trisaccharide side chain linked to C3 of every second glucose residue. The side chain is U-D-Man-(1->4)-U-D-GlcA-(1->2)-6-O-acetyl-alpha-D-Man-(1->beta-D-Man-(1->4)-beta-D-GlcA-(1->2)-alpha-D-Man-(1-> with approximately 60% of the terminal mannose units being pyruvylated and 90% of the proximal mannose units substituted at C6 with O-acetyl groups. It has side chains of 2 mannose and 1 gluconic glucuronic acid group.

Xanthan gum is an exocellular polysaccharide produced by fermentation of the bacteria Xanthomonas campestris, originally isolated from the rutabaga plant. It is a cream-coloured powder that is dissolved in water to produce a thick viscous solution at low concentrations. Xanthan remains stable over a wide temperature range and forms a strong film on drying.

Alginates are linear unbranched polymers naturally found in brown seaweeds (Phaeophyceae, mainly Laminaria) containing β-(1->4)-linked D-mannuronic acid (M) and α-(1->4)-linked L-guluronic acid (G) residues. Although these residues are epimers (D-mannuronic acid residues being enxymatically converted to L-guluronic after polymerization) and only differ at C5, they possess very different conformations; D-mannuronic acid being ⁴C₁ with diequatorial links between them and L-guluronic acid being ¹C₄ with diaxial links between them. Bacterial alginates are additionally O-acetylated on the 2 and/or 3 positions of the D-mannuronic acid residues. The bacterial O-acetylase may be used to O-acetylate the algal alginates, so increasing their water-binding.

Alginates are not random copolymers but, according to the source algae, consist of blocks of similar and strictly alternative residues (i.e. MMMMMM, GGGGGG, GMGMGMGM) each of which have differed conformational preferences and behaviour. They may be prepared with a wide range of average molecular weights (50-100000 residues) to suit the application.

“Designer” alginates can be generated by 5-epimerization of P-(1->4)-linked D-mannuronic acid residues to α-(1->4)-linked L-guluronic acid residues in algal alginates using bacterial epimerases. An available natural alternative is to harvest the seaweed from exposed seaboards (more G giving the kelp strength) or sheltered bays (more M).

An especially preferred sugar polymer of the present invention is polydextrose. The term “polydextrose” as used herein is a low calorie polymer of glucose that is resistant to digestion by the enzymes in the stomach. It includes polymer products of glucose which are prepared from glucose, maltose, oligomers of glucose or hydrolyzates of starch, or starch which are polymerized by heat treatment in a polycondensation reaction in the presence of an acid e.g. Lewis acid, inorganic or organic acid, including monocarboxylic acid, dicarboxylic acid and polycarboxylic acid, such as, but not limited to the products prepared by the processes described in the following U.S. Pat. Nos. 2,436,967, 2,719,179, 4,965,354, 3,766,165, 5,051,500, 5,424,418, 5,378,491, 5,645,647 or 5,773,604, the contents of all of which are herein incorporated by reference.

The term polydextrose also includes those polymer products of glucose prepared by the polycondensation of glucose, maltose, oligomers of glucose or starch hydrolyzates described hereinabove in the presence of a sugar alcohol, e.g. polyol, such as in the reactions described in U.S. Pat. No. 3,766,165. Moreover, the term polydextrose includes the glucose polymers, which have been purified by techniques described in the art, including any and all of the following but not limited to (a) neutralization of any acid associated therewith by base addition thereto, or by passing a concentrated aqueous solution of the polydextrose through an adsorbent resin, a weakly basic ion exchange resin, a type II strongly basic ion-exchange resin, mixed bed resin comprising a basic ion exchange resin, or a cation exchange resin, as described in U.S. Pat. Nos. 5,667,593 and 5,645,647, the contents of both of which are incorporated by reference; or (b) decolorizing by contacting the polydextrose with activated carbon or charcoal, by slurrying or by passing the solution through a bed of solid adsorbent or by bleaching with sodium chlorite, hydrogen peroxide and the like; (c) molecular sieving methods, like UF, RO (reverse osmosis), size exclusion, and the like; (d) or enzymatically treated polydextrose or (e) any other art recognized techniques known in the art.

Moreover, the term polydextrose includes hydrogenated polydextrose which, as used herein, includes hydrogenated or reduced polyglucose products prepared by techniques known to one of ordinary skill in the art. Some of the techniques are described in U.S. Pat. Nos. 5,601,863, 5,620,871 and 5,424,418, the contents of which are incorporated by reference.

Polydextrose is commercially available from companies such as Danisco, Staley and Shing Dong Bang.

In a preferred embodiment of the invention the polydextrose is hydrogenated polydextrose. It is preferred that the polydextrose used is purified. It may be made substantially pure using conventional techniques known to one skilled in the art, such as chromatography, including column chromatography, HPLC, and the like.

Especially for nutraceutical and pharmaceutical use it is more preferred that the polydextrose used is at least 80% pure, i.e. at least about 80% of the impurities are removed. More preferably it is at least 85% pure or even more preferably it is at least 90% pure. It is preferred that the polydextrose is non-hydrogenated polydextrose, hydrogenated polydextrose or non-hydrogenated polydextrose or hydrogenated polydextrose, which has been subject to purification or a mixture thereof.

In one embodiment of the present invention a composition is prepared by mixing a dose of a slowly fermented carbohydrate being effective in sustaining and controlling the fermentation throughout the colon with at least one nutritionally, nutraceutically or pharmacologically acceptable carrier and/or vehicle.

The carrier or vehicle may be any conventional compound used in the respective industry and which is compatible with the carbohydrate in question. The carrier may be solid, liquid or semiliquid. The solid may have any desired physical form conventionally used in edible and pharmaceutical products. The carrier may be inert in relation to the carbohydrate or it may have a beneficial effect of its own. Generally, the carbohydrates with or without carrier will be included in a pharmaceutical, nutraceutical or nutritional preparation as such. However, in some cases a coating such as an enteric coating may be provided on the carbohydrate in order to prevent its digestion prior to entering the colon.

A preferred carrier or vehicle to be mixed with polydextrose of the present invention to provide a composition is a polyol. The term “polyol” means hexitols such as sorbitol and mannitol, and pentitols such as xylitol. The term also includes C4-polyhydric alcohols such as erythritol or C12-polyhydric alcohols such as lactitol or maltitol. The polyol of the present invention is preferably selected from group comprising lactitol, xylitol, maltitol, sorbitol, isomalt. The most preferred polyol used in the present invention is lactitol. The weight ratio of polyol to polydextrose ranges preferably from about 1:10 to 10:1, more preferably from 1:5 to about 5:1.

In one preferred embodiment of the present invention the polyol is selected in such a way that it is effective to synergistically prevent the accumulation of lactic acid throughout the colon.

Other carriers and vehicles useful in the preparation of the present composition are edible and/or nutritional ingredients such as lactose, calcium and other minerals, vitamins, sugars and other components generally included into orally administrable compositions.

The composition of the present invention preferably comprises purified polydextrose and a polyol which is selected from the group consisting of lactitol, sorbitol, maltitol, xylitol and isomalt.

The slowly fermented carbohydrate, either alone or in synergistic effective amounts with a polyol, is administered to the subject in an amount effective to prevent the accumulation of lactic acid in the colon of the subject. As used here the term subject refers to animals, especially mammals, but also poultry and other animals having an intestine operating in a similar manner. Preferred animals include, but are not limited to human beings, pet animals (like dogs, cats, rodents, birds), farm animals (like horses, pigs, cattle, sheep, poultry), laboratory animals, zoo animals and other animals having a similar intestinal tract as those mentioned above. In the case of poultry the term “colon” should be taken to mean the caecum. Preferred poultry include hens, turkeys, pheasants, geese, etc.

The mammal of the present invention is more preferably a young mammal at the age of weaning, a young mammal suffering from milk crust, a mammal treated with antibiotics, a mammal having sensitivity to lactose, a mammal suffering from celiac disease, a mammal suffering from food allergy and/or an aged mammal. A composition according to the present invention is effective in alleviating the symptoms of disorders and diseases of these mammals having a risk of imbalanced fermentation.

The composition according to the present invention is preferably prepared in the form of an orally administrable preparation. The slowly fermented carbohydrate is administered orally to the subject in a composition which includes an effective dose of the slowly fermented carbohydrate and an edible carrier or vehicle. The preferred carrier is a polyol which has a synergistic effect on the carbohydrate.

The carbohydrate of the present invention is preferably added to a food product in effective amounts to sustain and control the fermentation throughout the colon of the subject, and the food containing the same is administered to the subject. It is also possible with the present invention to add the carbohydrate and polyol to a food product in synergistic effective amounts to prevent the accumulation of lactic acid throughout the colon of a mammal, when the food containing the same is administered to the mammal. It is also preferable to use the carbohydrates of the present invention in combination with probiotics.

In another preferred embodiment of the invention the carbohydrate and polyol are added to a food product in synergistic effective amounts to reduce the putrefactive fermentation in the colon of a subject, when the food containing the same is administered to the subject.

A preferred preparation of the present invention is a nutritional sour food (or feed) product. Compositions of the present invention are especially preferred in sour milk based preparations because the benefits of the carbohydrate are then obtained especially effectively. Preferred preparations are yogurt, baby's milk formula, sour milk, curdled milk, dry milk, crout (sauerkraut). It is also preferred to incorporate the carbohydrate of the present invention in meat products, such as sausages and meat balls. Further, the sustained release carbohydrates have beneficial effects in beverages such as health drinks or post-antibiotic alleviators.

The composition may be prepared in accordance with standard procedures for preparing pharmaceutically, therapeutically, nutritionally or nutraceutically acceptable compositions. Thus, the slowly fermented carbohydrate may be mixed with the carrier, e.g. with a polyol, and processed further into a dry, semi-dry or liquid product. The slowly fermented carbohydrate and polyol may also be granulated to provide a granulate which may be compressed into a tablet as such or with other common excipients and adjuvants, or added to a food or feed to be orally administered to a subject. A slowly fermented carbohydrate either alone or in combination with a polyol can be formulated to a capsule, tablet, pill or like by methods known in the art. The composition can also be formulated into a chewing gum or tablet, a powder, a spray, a syrup, a sugar substitute, a candy or sweet, a dairy product, a frozen dairy product, a pet food, an animal feed, and the like.

Preferred preparations are sweets and desserts which contain milk products such as chocolate and ice cream. Further, the present invention can be used to reduce the flatulence which many people note when chewing a chewing gum. Several benefits are obtained in baby foods and in milk crust alleviating preparations.

In a preferred embodiment of the invention the polydextrose is added to a food product in effective amounts to prevent the accumulation of lactic acid in the colon of a mammal, when the food containing the same is administered to the mammal. Food products are easily consumed and provide the effects of the composition to be obtained in a simple way.

Polydextrose is an ingredient designed to give the bulk, texture, mouthfeel and functional attributes of caloric sweeteners. A key to the prior art performance of polydextrose is its caloric value of 1 calorie per gram. Thus its is widely used as a calorie-reduced buLking agent in the dietetic food products.

The timing of the administration of the carbohydrate either alone or in synergistic effective amounts with a polyol is not critical and can be taken based upon individual needs. The efficient amount of the polydextrose for humans is approximately 1 g-100 g/day, preferably 5-50 g/day considering the individual differences. However, a regular daily use is recommended relating to food and feed intake and digestion, polydextrose should cover 0.1-10% of the daily diet, preferably 1-5%, most preferably 2-3%.

The following non-limiting examples further illustrate the invention.

### EXAMPlE 1

### Screening for Slowly Fermented Complex Carbohydrates

The following complex carbohydrates: polydextrose, xanthan, alginate, xylooligomer, starch, inulin, pectin, oligofructose and oligogalactose were screened for their properties in a fermentation by fecal bacteria. Both aqueous and crystalline compounds were used.

Feces from 3-4 donors were pooled and diluted with 5 parts of phosphate buffer at pH 7 with a reducing agent. The mixture was stirred anaerobically at 37° C. for 1 h and filtered to remove solid particles. 25 ml of filtrate and 0.25 g of each carbohydrate to be tested were mixed and incubated anaerobically at 37° C. with stirring (120 rpm) for 24 h. Gas production and pH were measured 1, 2, 3, 4, 12 and 24 hours after inoculation and microbes were measured at 12 and 24 hours after inoculation.

A slow increase in gas production and microbes as well as a slow decrease in the pH indicated that polydextrose, xanthan, alginate and xylooligomer were slowly fermented by the fecal bacterial. Starch, inulin, pectin, oligofructose and oligogalactose were rapidly fermented by the fecal bacteria, which was indicated by the high amount of gas in the early stages of the test and by a rapid decrease in the pH. Crystalline carbohydrates were fermented more slowly than aqueous ones.

### EXAMPLE 2

### Screening for Sustained Release Properties

Polydextrose (Litesse^{R} Ultra™, Danisco) and inulin (Raftiline^{R}, Orafti), two known prebiotics, were compared in a colon simulator for their effects on the gut microbial community throughout the colon. Polydextrose is a slowly fermented carbohydrate and inulin is rapidly fermented. Fresh fecal samples from five healthy human donors were pooled in anaerobic conditions and diluted in 0.9% anaerobic NaCl buffer. Fecal suspensions were then applied to the vessels of a 4-stage dynamic colon fermentation simulator. The computer-controlled simulator consists of four individual anaerobic vessels connected to each other with tubes, thus mimicking passage and fermentation of gut contents in the anaerobic conditions of the lower intestine. The simulation device provides detailed information on fermentation patterns of different prebiotics in different parts of the colon.

During the fermentation simulation a load of polydextrose and inulin, respectively, was added intermittently into the feed solution of the test prebiotic and applied to the first vessel (proximal part of the colon) of the system, and the contents of the vessels moved forward (towards more distal parts of the colon) in the simulator in three-hour cycles. The simulation was continued for 48 hours. The changes of pH were monitored during the run. After the completion of the run, samples of each stage were frozen and stored for later analyses. The concentrations of different volatile fatty acids and the degree of digestion were measured from the samples to determine fermentation patterns of the tested ingredients. 2% of polydextrose and 1% of inulin calculated on the weight of the feed solution mimicking normal nutrition were used for the study.

For the VFA-analysis 100 μl of sample solution, 100 μl of ISTD-solution (20 mM pivalic acid), 300 μl of water and 250 μl of saturated oxalic acid solution were mixed and allowed to stand for 60 min at 4° C. The samples were then centrifuged for 5 min at a maximum speed and 1 μl of the supernatant was injected into a gas chromatograph. The amount of inulin remaining in the sample was determined by size exclusion chromatography by comparing the peak areas before and after the simulation. The amount of polydextrose remaining in the sample was determined by using high pH anion chromatography and detected by pulsed electrochemical detector.

The production of volatile fatty acids in the two test runs is depicted in FIG. 1. The fermentation of polydextrose (PDX) resulted in the production of mainly acetic, propionic and butyric acids. The proportion of butyric acid increased towards the distal parts of the colon simulation. A substantial part of the polydextrose remained unfermented at the end of the study. In contrast, the fermentation of inulin was complete already in the first vessel. Also the fermentation patterns were different. The production of lactic acid was evident already after the first stage of the inulin test, and the concentration increased towards the distal parts of the colon simulation. In the polydextrose test no accumulation of lactic acid was seen at all indicating excellent sustained release properties.

### EXAMPLE 3

### Effect of Polydextrose

The effect of polydextrose on bacterial fermentation was studied in a clinical trial including healthy humans.

Daily doses of 10 g of polydextrose (Litesse^{R} Ultra™, Danisco) were ingested and the changes in the bacterial fermentation were measured from fecal samples obtained at 0, 3 and 6 weeks. Samples were stored at −20° C. prior to VFA measurements. VFA measurements were done as explained in Example 1. The concentration of branched VFAs decreased during the study (FIG. 2) but the concentration of butyrate increased concomitantly in relation to the decreasing proportion of branched VFAs (FIG. 3).

Polydextrose did not increase the putrefactive fermentation in humans. In fact, branched VFAs were decreased and butyrate production increased supporting the results from the colon simulation of Example 1 that polydextrose is a non-lactic acid producing carbohydrate which is effective in acting throughout colon. Polydextrose can be regarded as a balanced fermentation promoting prebiotic providing a sustained release of energy to the colon.

### EXAMPLE 4

### Yogurt Product

Pectin (Grindsted Pectin YF 310) 0.70% and crystalline lactitol 1.0% were blended dry and dissolved in water 11.0% that had been heated to 80-85° C. Raspberries (frozen) 50.0% polydextrose (Litesse® Ultra™, produced by Danisco Cultor America Inc.) 18.80% and crystalline lactitol 17.80% were heated to boil and after that the mixture of pectin and lactitol was added while agitating well. A calcium slurry was made by dissolving a calcium salt (Calcium lactate 5 H₂O) 0.296% in hot water 5.0%, and then it was added to the fruit mass, while agitating well. The mixture was then evaporated until the desired content was reached. pH was adjusted to 3.9 using sodium citrate solution, and preservatives (K-sorbate 20% w/v, 0.25%) were added. For filling the mixture was cooled to temperature of 40° C. The mixture was dosed into a yogurt at a final dosage of 15-20%. Ingestion of one to two yogurts per day provided a balanced and healthy intestinal function without flatulence or indications of lactic acid accumulation. The percentages are calculated on fresh weight basis.

### EXAMPLE 5

### Baby's Milk Formula

A standard baby's milk formula containing milk protein is mixed with 2.5% by weight of polydextrose dissolved in distilled water. The mixture is agitated until uniform and filled in 2dl packages and sterilized to provide a baby feed for reducing colic and milk crust.

### EXAMPLE 6

### Sausages

Frankfurter sausages are prepared according to a standard sausage recipe with the exception that polydextrose (Litesse® II, Danisco) at a dose of 10% by weight of the sausage mass is mixed into the mass prior to stuffing. The resulting sausages have a good consistency and taste and they are suitable for ingestion by people with a sensitive stomach.

### EXAMPLE 7

### Pharmaceutical Formulation

A tablet containing polydextrose (Litesse® Ultra, Danisco) and lactitol (Lactitol Monohydrate, Danisco) is prepared by granulating the lactitol with the polydextrose. The resulting granules are mixed with magnesium stearate and compressed into tablets for use in the prophylaxis of acidosis.

### EXAMPLE 8

### Post-Antibiotic Beverage

A composition for alleviating stomach disorders after an intake of antibiotics is produced by mixing 60% of granulated lactic acid bacteria and 40% alginate. Before ingestion the composition is mixed in a glass of water.

### EXAMPLE 9

### Cocoa Drink

A composition of lactitol monohydrate (200 g), fat free milk powder (70 g), low fat cocoa powder (12 g) and polydextrose (100 g) is produced by mixing the ingredients in dry form. For use the composition is mixed into hot water (700 g) to provide a hot health drink.

Unless indicated to the contrary, the percentages are weight percentages. Moreover, the weights provided are the dry weights, i.e., excluding the weight of the carrier, which may be present.

The above preferred embodiments and examples are given to illustrate the scope and spirit of the present invention. These embodiments and examples will make apparent to those skilled in the art other embodiments and examples. These other embodiments and examples are within the contemplation of the present invention.

1. A method for the therapeutic or prophylactic treatment of subjects suffering from or being subject to a risk of imbalanced colon fermentation said method comprising administering slowly fermented complex oligomeric or polymeric carbohydrate(s) to said subject in an amount which is effective in sustaining and controlling the fermentation throughout the colon of said subject.

2. The method according to claim 1, wherein said carbohydrate is a sugar polymer.

3. The method according to claim 2, wherein said sugar polymer is polydextrose.

4. The method according to claim 1, wherein said carbohydrate is administered in an amount which is effective in preventing the accumulation of lactic acid throughout the colon.

5. The method according to claim 1, wherein said carbohydrate is administered in an amount which is effective in reducing the pH throughout the colon without accumulation of lactic acid.

6. The method of according to any one of claims 1 to 5, wherein said carbohydrate is administered in an amount which is additionally effective in reducing the putrefactive fermentation throughout the colon.

7. The method of according to any one of claims 1 to 6, wherein said carbohydrate is administered in an amount which is additionally effective in increasing the amount of butyrate throughout the colon.

8. The method according to any one of claims 1 to 3, wherein said carbohydrate is administered in an amount which is effective in increasing the tolerance of probiotic lactic acid bacteria.

9. The method according to any one of claims 1 to 3, wherein said carbohydrate is administered in an amount which is effective in facilitating the management of lactose intolerance.

10. The method according to any one of claims 1 to 3, wherein said carbohydrate is administered in an amount which is effective in facilitating the management of food allergy.

11. The method according to any one of claims 1 to 3, wherein said carbohydrate is administered in an amount which is effective in facilitating the management of celiac disease.

12. The method according to any one of claims 1 to 3, wherein said carbohydrate is administered in an amount which is effective in reducing the risk of inflammatory diseases in the colon.

13. The method according to any one of claims 1 to 3, wherein said carbohydrate is administered in an amount which is additionally effective in balancing or normalizing the microbial community throughout the colon.

14. The method according to any one of claim 1 to 3, wherein said carbohydrate is administered in combination with at least one nutritionally, nutraceutically and/or pharmacologically acceptable carrier and/or vehicle.

15. The method according to claim 14, wherein said carrier and/or vehicle is a polyol.

16. The method according to claim 15, wherein said carbohydrate and said polyol are administered in synergistic effective amounts to prevent the accumulation of lactic acid throughout the colon.

17. The method according to claim 15 or 16, wherein said polyol is selected from the group comprising lactitol, xylitol, maltitol, sorbitol, isomalt.

18. The method according to claim 17, wherein said polyol is lactitol.

19. The method according to any one of claims 1 to 18, wherein said carbohydrate is administered to a subject selected from the group consisting of human beings, pet animals, farm animals, laboratory animals, zoo animals.

20. The method according to claim 19, wherein said subject is selected from the group consisting of a young mammal at the age of weaning, a young mammal suffering from milk crust, a mammal treated with antibiotics, a mammal having sensitivity to lactose, a mammal suffering from celiac disease, a mammal suffering from food allergy and an aged mammal.

21. The method according to any one of claims 1 to 20, wherein said carbohydrate is incorporated into a composition to be administered orally.

22. The method according to any of the claims 1 to 21, wherein said composition is prepared in the form of an orally administrable preparation selected from the group comprising a dry, semidry or liquid food product, a tablet, a pill, a chewing gum or tablet, a powder, a spray, a syrup, a sugar substitute, a candy or sweet, a dairy product, a frozen dairy product, a meat product, a health drink, a baby food, a pet food, an animal feed, and the like.

23. The method according to claim 22, wherein said preparation is a sour food or feed product, preferably a sour milk product.

24. The method according to claim 23, wherein said preparation is a sour milk product

25. The method according to claim 22, wherein said preparation is selected from the group consisting of yogurt, baby's milk formula, sour milk, curdled milk, dry milk and crout.

26. The method according to claim 3, wherein said polydextrose is hydrogenated polydextrose.

27. The method according to claim 3, wherein said polydextrose is purified.

28. The method according to claim 26 or 27, wherein said polydextrose is selected from the group consisting of non-hydrogenated polydextrose, hydrogenated polydextrose, andnon-hydrogenated polydextrose or hydrogenated polydextrose, which has been subject to purification and a mixture thereof.

29. The method according to claim 1, wherein said carbohydrate selected from the group consisting of xanthan, alginate and a xylooligomer.

30. The method according to claim 15, wherein the weight ratio of polyol to polydextrose ranges from about 1:10 to 10:1, preferably from 1:5 to about 5:1.

31. The method according to claim 21 or 22, wherein the carbohydrate is added to a food product in effective amounts to sustain and control the fermentation throughout the colon of a mammal, and the food containing the same is administered to the mammal.

32. The method according to claim 21, wherein the carbohydrate and polyol are added to a food product in synergistic effective amounts to prevent the accumulation of lactic acid throughout the colon of a mammal, and the food containing the same is administered to the mammal.

33. The method according to claim 21, wherein the carbohydrate and polyol are added to a food product in synergistic effective amounts to reduce the putrefactive fermentation in the colon of a mammal, when the food containing the same is administered to the mammal.
